SEQUENCE LISTING (11 sequences)

SEQ ID NO: 1 - DNA, 504 bp, synthetic construct
  source 1..504: mol_type = other DNA
atgtccgaggtggagttctcccacgagtactggatgaggcacgccctcaccctcgccaagagggccagggacgagagggaggtgccagtgggcgccgtgctcgtgctcaacaacagggtgatcggcgagggctggaacagggccatcggcctccacgacccaaccgcccacgccgagatcatggccctcaggcaaggcggcctcgtgatgcaaaactacaggctcatcgacgccaccctctacgtgaccttcgagccatgcgtgatgtgcgccggcgccatgatccactccaggatcggcagggtggtgttcggcgtgaggaactccaagaggggcgccgccggctccctcatgaacgtgctcaactacccaggcatgaaccacagggtggagatcaccgagggcatcctcgccgacgagtgcgccgccctcctctgcgacttctacaggatgccaaggcaagtgttcaacgcccaaaagaaggcccaatcctccatcaactga

SEQ ID NO: 2 - DNA, 4107 bp, synthetic construct
  source 1..4107: mol_type = other DNA
atggacaagaagtactccatcggcctcgccatcggcaccaactccgtgggctgggccgtgatcaccgacgagtacaaggtgccatccaagaagttcaaggtgctcggcaacaccgacaggcactccatcaagaagaacctcatcggcgccctcctcttcgactccggcgagacggccgaggccaccaggctcaagaggaccgccaggaggaggtacaccaggaggaagaacaggatctgctacctccaagagatcttctccaacgagatggccaaggtggacgactccttcttccacaggctcgaggagtccttcctcgtggaggaggacaagaagcacgagaggcacccaatcttcggcaacatcgtggacgaggtggcctaccacgagaagtacccaaccatctaccacctcaggaagaagctcgtggactccaccgacaaggccgacctcaggctcatctacctcgccctcgcccacatgatcaagttcaggggccacttcctcatcgagggcgacctcaacccagacaactccgacgtggacaagctcttcatccaactcgtgcaaacctacaaccaactcttcgaggagaacccaatcaacgcctccggcgtggacgccaaggccatcctctccgccaggctctccaagtccaggaggctcgagaacctcatcgcccaactcccaggcgagaagaagaacggcctcttcggcaacctcatcgccctctccctcggcctcaccccaaacttcaagtccaacttcgacctcgccgaggacgccaagctccaactctccaaggacacctacgacgacgacctcgacaacctcctcgcccaaatcggcgaccaatacgccgacctcttcctcgccgccaagaacctctccgacgccatcctcctctccgacatcctcagggtgaacaccgagatcaccaaggccccactctccgcctccatgatcaagaggtacgacgagcaccaccaagacctcaccctcctcaaggccctcgtgaggcaacaactcccagagaagtacaaggagatcttcttcgaccaatccaagaacggctacgccggctacatcgacggcggcgcctcccaagaggagttctacaagttcatcaagccaatcctcgagaagatggacggcaccgaggagctgctcgtgaagctcaacagggaggacctcctcaggaagcaaaggaccttcgacaacggctccatcccacaccaaatccacctcggcgagctgcacgccatcctcaggaggcaagaggacttctacccattcctcaaggacaacagggagaagatcgagaagatcctcaccttccgcatcccatactacgtgggcccactcgccaggggcaactccaggttcgcctggatgaccaggaagtccgaggagacgatcaccccatggaacttcgaggaggtggtggacaagggcgcctccgcccaatccttcatcgagaggatgaccaacttcgacaagaacctcccaaacgagaaggtgctcccaaagcactccctcctctacgagtacttcaccgtgtacaacgagctgaccaaggtgaagtacgtgaccgagggcatgaggaagccagccttcctctccggcgagcaaaagaaggccatcgtggacctcctcttcaagaccaacaggaaggtgaccgtgaagcaactcaaggaggactacttcaagaagatcgagtgcttcgactccgtggagatctccggcgtggaggacaggttcaacgcctccctcggcacctaccacgacctcctcaagatcatcaaggacaaggacttcctcgacaacgaggagaacgaggacatcctcgaggacatcgtgctcaccctcaccctcttcgaggacagggagatgatcgaggagaggctcaagacctacgcccacctcttcgacgacaaggtgatgaagcaactcaagaggaggaggtacaccggctggggcaggctctccaggaagctcatcaacggcatcagggacaagcaatccggcaagaccatcctcgacttcctcaagtccgacggcttcgccaacaggaacttcatgcaactcatccacgacgactccctcaccttcaaggaggacatccaaaaggcccaagtgtccggccaaggcgactccctccacgagcacatcgccaacctcgccggctccccagccatcaagaagggcatcctccaaaccgtgaaggtggtggacgagctggtgaaggtgatgggcaggcacaagccagagaacatcgtgatcgagatggccagggagaaccaaaccacccaaaagggccaaaagaactccagggagaggatgaagaggatcgaggagggcatcaaggagctgggctcccaaatcctcaaggagcacccagtggagaacacccaactccaaaacgagaagctctacctctactacctccaaaacggcagggacatgtacgtggaccaagagctggacatcaacaggctctccgactacgacgtggaccacatcgtgccacaatccttcctcaaggacgactccatcgacaacaaggtgctcaccaggtccgacaagaacaggggcaagtccgacaacgtgccatccgaggaggtggtgaagaagatgaagaactactggaggcaactcctcaacgccaagctcatcacccaaaggaagttcgacaacctcaccaaggccgagaggggcggcctctccgagctggacaaggccggcttcatcaagaggcaactcgtggagacgaggcaaatcaccaagcacgtcgcccaaatcctcgactccaggatgaacaccaagtacgacgagaacgacaagctcatcagggaggtgaaggtgatcaccctcaagtccaagctcgtgtccgacttcaggaaggacttccaattctacaaggtgagggagatcaacaactaccaccacgcccacgacgcctacctcaacgccgtggtgggcaccgccctcatcaagaagtacccaaagctcgagtccgagttcgtgtacggcgactacaaggtgtacgacgtgaggaagatgatcgccaagtccgagcaagagatcggcaaggccaccgccaagtacttcttctactccaacatcatgaacttcttcaagaccgagatcaccctcgccaacggcgagatcaggaagaggccactcatcgagacgaacggcgagacgggcgagatcgtgtgggacaagggcagggacttcgccaccgtgaggaaggtgctctccatgccacaagtgaacatcgtgaagaagaccgaggtgcaaaccggcggcttctccaaggagtccatcctcccaaagaggaactccgacaagctcatcgccaggaagaaggactgggacccaaagaagtacggcggcttcgactccccaaccgtggcctactccgtgctcgtggtggccaaggtggagaagggcaagtccaagaagctcaagtccgtgaaggagctgctcggcatcaccatcatggagaggtcctccttcgagaagaacccaatcgacttcctcgaggccaagggctacaaggaggtgaagaaggacctcatcatcaagctcccaaagtactccctcttcgagctggagaacggcaggaagaggatgctcgcctccgccggcgagctgcaaaagggcaacgagctggccctcccatccaagtacgtgaacttcctctacctcgcctcccactacgagaagctcaagggctccccagaggacaacgagcaaaagcaactcttcgtggagcaacacaagcactacctcgacgagatcatcgagcaaatctccgagttctccaagagggtgatcctcgccgacgccaacctcgacaaggtgctctccgcctacaacaagcacagggacaagccaatcagggagcaagccgagaacatcatccacctcttcaccctcaccaacctcggcgccccagccgccttcaagtacttcgacaccaccatcgacaggaagaggtacacctccaccaaggaggtgctcgacgccaccctcatccaccaatccatcaccggcctctacgagacgaggatcgacctctcccaactcggcggcgactga

SEQ ID NO: 3 - DNA, 54 bp, synthetic construct
  source 1..54: mol_type = other DNA
aagaggaccgccgacggctccgagttcgagtccccaaagaagaagaggaaggtg

SEQ ID NO: 4 - DNA, 96 bp, synthetic construct
  source 1..96: mol_type = other DNA
tccggcggctcctccggcggctcctccggctccgagacgccaggcacctccgagtccgccaccccagagtcctccggcggctcctccggcggctcc

SEQ ID NO: 5 - DNA, 48 bp, synthetic construct
  source 1..48: mol_type = other DNA
aagaggccagccgccaccaagaaggccggccaagccaagaagaagaag

SEQ ID NO: 6 - DNA, 4101 bp, synthetic construct
  source 1..4101: mol_type = other DNA
gacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgac

SEQ ID NO: 7 - DNA, 66 bp, synthetic construct
  source 1..66: mol_type = other DNA
gattacaaggaccacgacggggattacaaggaccacgacattgattacaaggatgatgatgacaag

SEQ ID NO: 8 - DNA, 51 bp, synthetic construct
  source 1..51: mol_type = other DNA
atggctccgaagaagaagaggaaggttggcatccacggggtgccagctgct

SEQ ID NO: 9 - DNA, 48 bp, synthetic construct
  source 1..48: mol_type = other DNA
aagcggccagcggcgacgaagaaggcggggcaggcgaagaagaagaag

SEQ ID NO: 10 - DNA, 1281 bp, synthetic construct
  source 1..1281: mol_type = other DNA
atggagagaataagcaccaatcagctctacaattctggaattccggtgactgtgccatcgcctctgcctgctataccagctaccctggatgaaaacattcccaggattccagatgggcagaatgttccgcgggagagagaattgagaagcacacctatgccacctcatcagaatcagagtactgttgctcctcttcatgggcattttcagtccagtaccgggtctgttgggcctctgcgttcgtcccaggcgataaggttctcttcagtttcaagcaatgagcaatatacaaatgccaatccttacaattctcaaccgccgagtagtgggagttcttcaacgctcaattatggatcacaatatggaggctttgaaccttccttgactgattttccaagagatgctgggccgacgtggtgtcctgatccagttgatggcttgcttggatatacagatgatgtccctgctgggaacaatttgactgaaaacagttctattgcagctggtgatgaacttgccaagcaaagtgaatggtggaatgattttatgaattatgactggaaagatattgataacacagcttgtactgaaactcaaccacaggttggaccagctgcgcaatcatctgtcgcagttcaccaatcagctgcccaacaatcagtttcatctcaatcaggagaaccttctgcagttgctataccctcgccctctggtgcctccaatacctccaactccaagacacgaatgagatggactcctgaacttcatgagcgctttgtagatgctgtcaatctacttggtggcagtgaaaaagctactcccaagggtgtgttaaagctaatgaaggcagacaatttgaccatttatcatgttaaaagtcaccttcagaaatacagaacagctcgatacagaccagaattgtctgaaggttcttcagaaaagaaggcagcctcaaaagaggacataccatcaatagatctgaaaggagggaactttgatctcactgaggcattgcgtctccagttagaactccaaaagaggcttcatgaacagcttgagatccaaagaagtttgcagctgagaattgaggagcaagggaagtgccttcagatgatgctcgagcagcagtgcatacctgggacagacaaggcggtggatgcttcaacctcagcagaaggaacaaagccatcttctgatcttccagaatcttctgccgtgaaggatgttccagagaacagtcagaacggaatagccaaacaaacagaatcaggtgacagataa

SEQ ID NO: 11 - DNA, 2561 bp, synthetic construct
  source 1..2561: mol_type = other DNA
ccaattagcccagcctggtgttaattagctggatgactggatcttactatacatggcaaaagtgttcaccactttgatgtcaattattggagagttaattacccatatatatgcgtagtatatgtgattttgaaagtgtccaaacatgtagtgcaattttattgggagtaattaatacactgaattaaaattcataaaagaaagataaggtgttaccaggtcagagattttactttacttaaataccacatagcaatgtgaatacgtgtggtgaaactataccactttgatttatggacaaagttactgatgatagttacactaaaactaaataatgcaatcaacatggcctcagtaacatggataaaaaactactaaattattattgccgaaagtaattgggtgacttcgtcaagatcttactgttgtacgtgaagtgtgaacagtaccgtaccgtctaattttataaaggatgcagcgtgagacgggtatattaaccactaactcgcactaggacggcttatcaaccatttacaataaagcattaaagccttcttcatagtggagaaatgtgaaagcacttttaaagaaattacgccaaactatataaaattcttacgttgtaagaagccccaaatatgtatgattcactgattcacacagcattggatgatgatttagatctctctgatttaagttaggtgactttaaagacactaacatgtggaagatatggatccttccttttcctcgtaataaaccatcacataaataaaactaaccatcctaaagcctcaacaatcgtgaaaaactgtagatatagttcttggaaaattcatatctttctttcggaattacaaaactagaaaaaaaatactcccatcgttttaaaatataagtatttctggttatgaatctggacaagtgtttatctagattcatagttaaaagttgttatattttaagataatgtagtgcttattagaaagacattacatcttttccacaaagacttttctttttttactatgaatttgaataagtatttctctaggtggatatcctaaaatgaaatactctattcgtctcaaatatagcaacttaatacaacattagacaccacttattaatatgaatctggatagggataacgaatctagacatgattcatggcactaggttatatctattttattttagttaccgttatagtaccttctctatcttaaaaaacaaatcatgttcagatttatagcactgggatgcatcacatcccgtagtagtttatttttatgggacgaaaagagcacatcagaatcatgtgctttgaaaaagatcaaaaacaaaaaaaaagaacatccaaaggcaaattccttcttgggtacaaccatgtactctagtcctacaaagtaccacataattcttgccacttgccatctcttccctctccctccccatttgttcgattccccatttggccttttcctagaaccatcctccctcccccacaaaaccccccaaaaaaattacaacaaaagcaaaatggatttgaacaaaattcaggatgaaaccttgaattcaacactgcaccctcctactagtagtagcacctctaccagttacttctcaatccgtaccaaaatataaacacttctaaaataatatcaagccaaatattttttaactttgattattaatagaaaaaaaataaaaacaaatcaatcatgtaaaattgatatttactagatttatcattaaacaactatcatgctccatatgtaactttttttattttaaacatcgtacttttatagatattattagtcaaagtagtatctcgaagactaagtgtaaaattgtttatattttagagcggggagagagagctacccatcttcatcagctaatgatccaaaagaggcaccaaaaagaagaaggaagaaaaaaacacgaaacgcgcagtcgcgtctcacccccatttgccgcacgttgcccaactcctcctcctcctcgtcatcgtctccgttccgatccgcgcccataaatacgcgccaccccgcccccaacctcgccgtccttgtcccccccaagaaccccccgtgcgccaccaccaccaccaccaccaccaccaccaccaccgaggaattctcgctgtcgccgccgccgacgacgacgaggagaaggagtatcgctcacaatcttccgggccgatggggagggcgccgtgctgcgagaaggaggggctgaggagaggggcgtggagccccgaggaggacgaccgcctcgtcgcctacatccgccgccacggccaccccaactggcgcgcgctccccaagcaagccggttagtagtagcctccgccgccgccgccgccgccgttgctgttgttcttgggttgatgatgatgatgagatgagatcggtgttggttggttgcagggcttctccgctgcgggaagagctgcaggctgcggtggatcaactacctccggccggacatcaagcgggggaacttcaccgccgacgaggaggacctcatcgtccgcctccacaactccctcg

(19) United States
(12) Patent Application Publication
(10) Pub. No.: US 2026/0155211 A1
(43) Pub. Date: Jun. 4, 2026

(54) PROTEIN ENGINEERING AND DIRECTED EVOLUTION METHOD BASED ON GRAPH DEEP LEARNING AND APPLICATIONS THEREOF

(71) Applicant: Henan Agricultural University, Zhengzhou (CN)

(72) Inventors: Xiang JI, Zhengzhou (CN); Zhen CHEN, Zhengzhou (CN); Zhaohui QIN, Zhengzhou (CN); Xiaomin SI, Zhengzhou (CN)

(73) Assignee: Henan Agricultural University, Zhengzhou, HA (CN)

(21) Appl. No.: 19/532,228

(22) Filed: Feb. 6, 2026

(30) Foreign Application Priority Data
Sep. 5, 2025 (CN) 202511263086.1

(51) Int. Cl. G16B 40/20; G06N 3/042; G06N 3/0895; G16B 15/00

(52) U.S. Cl. CPC G16B 40/20; G06N 3/042; G06N 3/0895; G16B 15/00

(57) ABSTRACT

The present disclosure belongs to the field of computational biology and protein engineering technology. The present disclosure provides a protein engineering and directed evolution method based on graph deep learning and applications thereof, the method includes the following steps: S1, construction of a protein structural dataset; S2, protein graph representation; S3, graph neural network model architecture; S4, model training and performance evaluation; S5, model inference, and finally identification of potential mutations that can improve the fitness. The present disclosure can realize zero-shot, low-cost, high-efficiency, and accurate prediction of protein variants with improved properties; meanwhile, TadA8ePro with improved A-to-G base editing efficiency, Cas9Plus with higher gene knockout efficiency, and OsPHR2 transcription factor with improved binding activity are also provided. The present disclosure realizes the rapid, low-cost, and efficient engineering of genome editing proteins and transcription factors, and provides a powerful tool for accelerating crop breeding and synthetic biology.

### TECHNICAL FIELD

The present disclosure belongs to the field of computational biology and protein engineering technology, and specifically relates to a protein engineering and directed evolution method based on graph deep learning and applications thereof.

### BACKGROUND

As the terminus of central dogma of molecular biology, proteins perform specific biological functions through their unique amino acid sequences and three-dimensional structures. The diversity and specificity of protein sequence space enables them to play irreplaceable roles in living organisms, such as catalyzing biochemical reactions via enzymes, mediating immune responses through antibodies, and regulating physiological processes via hormones. Moreover, proteins participate in intercellular communication, signal transport, and the regulation of gene expression, making them indispensable molecules for sustaining life activities. Accordingly, protein engineering aimed at optimizing protein function is of great biological and industrial significance. To develop more efficient proteins, researchers typically employ methods such as deep mutational scanning (DMS), directed evolution, and structure-based rational design. Both DMS and directed evolution require screening numerous mutants through wet-lab experiments, which are time-consuming, labor-intensive, and costly. Structure-based rational design relies on accurate determination of protein three-dimensional structures, such as by X-ray crystallography or cryo-electron microscopy, which involves highly specialized procedures and extensive experimental efforts. Therefore, there is a strong demand for a low-cost, efficient, and scalable protein engineering method capable of optimizing protein functions.

Recent advances in artificial intelligence, especially in machine learning (ML), have brought transformative progress to the field of protein engineering. Among existing computational approaches, protein language models have emerged as a widely adopted technique. These models can learn evolutionary patterns from hundreds of millions to billions of protein sequences via masked language modeling, thereby enabling efficient exploration of protein sequence space. With the aid of open-source frameworks such as ESM and ProtTrans, beneficial mutations can be identified more rapidly, significantly reducing experimental screening costs. However, training language models from scratch requires substantial computational resources, which limits their accessibility and scalability. More importantly, protein function is fundamentally determined by its three-dimensional structure and interactions with other biomolecules. Language models trained solely on protein sequences lack explicit structural and spatial information, making it difficult to accurately capture mutations that affect protein folding, stability, and molecular interactions. Consequently, sequence-only language models exhibit limited capability in predicting structure-dependent functional changes. Therefore, there remains a pressing need for a low-cost, efficient, and structure-aware deep learning method capable of accurately screening protein variants with enhanced functional outcomes.

Compared with traditional neural networks, graph neural networks are well-suited for modeling protein structures because of their advantages including expressiveness, permutation invariance and scalability. Graph neural networks can not only capture local structural information of proteins, such as interactions between adjacent amino acids, but also model global relationships, such as interactions among higher-order neighbors, through information aggregation and multi-layer network updates.

In recent years, genome editing technologies based on the CRISPR/Cas system and its derivatives have greatly accelerated the functional analysis and targeted molecular breeding of crop traits, owing to their simplicity, efficiency, and precision. These tools enable effective gene knockout and deletion, transcriptional regulation, single-base editing, and insertion or replacement of DNA fragments in crops. Through targeted genetic modifications, they have demonstrated broad application potentials in the enhancement of valuable crop traits including stress resistance, yield, and quality, and the study of functional genomics. However, implementing these functions relies on the development of diverse genome editing systems, and optimizing the efficiency of different systems requires extensive time-consuming, laborious, and costly wet experiments. Modifying functional proteins within editing systems represents an important approach to optimizing and improving the efficiency of gene editing technology, with deep learning-assisted directed evolution offering a simple and efficient strategy for the optimization of more efficient gene editing tools.

As a bridge between genomic information and cellular function, the regulatory role of transcription factors spans from stress responses in single-celled organisms to advanced neural activities in humans, and from embryonic development to disease treatment. Their importance is irreplaceable in both basic biology and biotechnology. Plant transcription factors play a central role in regulating plant growth and development, responding to abiotic stress, and resisting biotic stress. For example, the rice transcription factor OsPHR2 belongs to the MYB transcription factor family, possesses transcriptional activation activity, and mediates signal transduction under phosphorus starvation conditions. Studies have shown that plants overexpressing OsPHR2 exhibit a dwarf phenotype, while OsPHR2-deficient mutants display a taller plant stature. Through deep learning-assisted directed modification, mutants capable of enhancing the DNA-binding activity of transcription factors to downstream target gene promoters can be screened, offering a promising approach to improving rice lodging resistance.

Therefore, in view of the aforementioned limitations in existing protein engineering approaches, it is necessary to propose a protein engineering and directed evolution method based on graph deep learning based a meta-learning fine-tuning strategy.

### SUMMARY

In order to solve the problems in background technology, the present disclosure provides a protein engineering and directed evolution method based on graph deep learning, which realizes zero-shot, low-cost, efficient, and accurate prediction of protein variants with improved protein activity and efficiency.

TadA8ePro (TadA8e-T83N) with improved editing efficiency, Cas9Plus (Cas9-D1180G) with higher editing efficiency, and OsPHR2 (H294R) transcription factor with improved binding activity are also provided.

In order to achieve the above purpose, the technical scheme of the present disclosure is as follows:

The content is the same as the claims and is temporarily omitted.

The beneficial effects of this application:

For a protein with a length of L, the theoretical sequence space can reach 20^{L}. Even when only considering single point mutations, the number of possible variants is as high as is L×19. Exhaustively validating such variants through experimental approaches would require enormous investments of time, labor, and material resources, rendering comprehensive exploration of the sequence space impractical. The method disclosed herein, for the first time, integrates geometric deep learning model with a meta-learning strategy to enable efficient and accurate screening of beneficial mutations. By leveraging structural representation learning and rapid task adaptation, the proposed method can reduce the candidate single point mutations from up to L×19 to only dozens or even a few highly promising variants. Meanwhile, it can still nominate mutations that significantly improve activity or efficiency in such a limited candidate set. Compared with the conventional protein engineering methods relying on large-scale random mutagenesis, the present disclosure significantly lowers experimental workload and cost, while achieving substantial improvements in desired properties, computational/experimental efficiency and high success rate. Accordingly, the proposed method represents a notable technological advancement and provides strong practical value and innovation for protein engineering.

The present disclosure successfully engineered three proteins: 1. TadA8ePro (TadA8e-T83N) variant, increasing A-to-G base editing efficiency 1.54-2.24 fold in wheat; 2. Cas9Plus protein, with the editing efficiency achieving 9.07-fold in multiple endogenous gene loci of wheat; 3. Rice OsPHR2 transcription factor, with the binding affinity of H294R variant 4.6-fold higher than the wild type.

The present disclosure realizes the rapid, low-cost, and efficient function enhancement of gene-edited proteins and transcription factors, and provides a powerful tool for accelerating breeding and synthetic biology.

### BRIEF DESCRIPTION OF THE DRAWINGS

FIG. 1 is an overview of the neural network algorithm;
where A represents the data preprocessing process; B is the implementation of the proposed graph neural network model;

FIG. 2 is a diagram of meta-learning strategy for inferring family-specific fitness landscapes;
where A is the input module and data processing flow, including target protein input, homologous protein database construction, and protein clustering; B is the model training and prediction process, including meta-learning dataset split, model fine-tuning, and beneficial mutation prediction;

FIG. 3 is a performance comparison of different fine-tuning strategies and pre-training models;
where A is the sequence recovery rate of different fine-tuning strategies and pre-training models; B is the confusion of different fine-tuning strategies and pre-training models;

FIG. 4 is a meta-learning fine-tuning model improves the folding performance of the model in specific proteins;
where A is the folding performance comparison between the pre-trained model and the fine-tuned model of TadA8e (PDB ID: 6VPC strand E); B is the folding performance comparison between the pre-training model and the fine-tuning model of TadA8e (PDB ID: 6VPC strand F); C is the folding performance comparison between the pre-training model and the fine-tuning model of SpCas9 (PDB ID: 4008 strand D). D is the comparison of folding performance between the OsPHR2 (AlphaFold prediction structure) pre-training model and fine-tuning model. Note: The following text in each subgraph represents the folding performance of the pre-trained model and the fine-tuned model, respectively. The comparison indicators include i) Sequence Recovery: The percentage of the predicted sequence that matches the real sequence. A higher value indicates more accurate sequence prediction. ii) RMSD (Root Mean Square Deviation): It measures the spatial deviation between the predicted structure and the reference structure. A smaller value indicates that the predicted structure is closer to the true folding. iii) Average pLDDT (predicted Local Distance Difference Test): A confidence score for the overall predicted protein structure. A higher value reflects a more reliable predicted structure.

FIG. 5 is shows predicted mutations in TadA8e structure;
where A is the molecular surface visualization structure of SpCas9 and TadA8e complex. The left and right diagrams represent the views before and after 180° rotation around the Y axis, respectively. In the diagram, Cas9 is shown in green, DNA is shown in blue, gRNA is shown in orange, and the two dimers of TadA8e are shown in pink; B is a schematic diagram of the spatial position of the predicted mutant residues in the protein structure of TadA8e, and the mutant residues are represented by a cyan stick model;

FIG. 6 is a TadA8e expression vector diagram;
where A is the protein expression vector B-UBI-TadA8e-SpCas9n-NOS; B is the relative position of the single amino acid mutation site of TadA8e; C is the guide RNA expression vector B-TaU3-tRNA-sgRNA-tRNA;

FIG. 7 is a comparison of the editing efficiency of different variants of TadA8e;
where A is the result of flow cytometry analysis of TadA8e variant; B is the editing efficiency of TadA8e variant at three endogenous sites of TaATX4, TaGW8 or TaDEP1;

FIG. 8 shows predicted mutations in SpCas9 structure;
where A is the molecular surface visualization map of the SpCas9 crystal structure; the left and right diagrams represent the views before and after 180° rotation around the Y axis, respectively. In the diagram, the functional domains RuvC, BH, REC, and PI are displayed in green, cyan, gray, and orange, respectively. B is a spatial position diagram of the mutant residue in the protein structure predicted in SpCas9, and the mutant residue is expressed in the form of a pink stick model.

FIG. 9 is a SpCas9 expression vector diagram;
where A is the protein expression vector B-UBI-SpCas9-NOS; B is the guide RNA expression vector B-TaU3-tRNA-sgRNA-tRNA-polyT; C is the relative position of SpCas9 single amino acid mutation site;

FIG. 10 is a comparison of editing efficiency of SpCas9 mutants at TaLOX2, TaPIN1 and TaGW2 loci;

FIG. 11 shows predicted mutations in OsPHR2;

FIG. 12 is an OsPHR2 expression vector diagram;

FIG. 13 is the LUC screening results of OsPHR2 mutants, with wild type (WT) as a reference, the activation activity of other variants is expressed as a difference multiple relative to the wild type, which is convenient for visually comparing the effect of different mutations on the activation activity.

### DETAILED DESCRIPTION OF THE EMBODIMENTS

The following is a clear and complete description of the technical solution of the present disclosure in conjunction with the accompanying drawings. Any equivalent replacements or modifications made to the present disclosure by those of ordinary skill in the art without departing from its concept and technical solution shall fall within the scope of protection of the present disclosure.

The protein engineering and directed evolution method based on graph deep learning includes the following steps:

S1, a protein structure dataset is constructed: The PDB50 dataset is collected using a PISCES server.

The specific screening criteria are as follows: 1.1, the structure determination methods include X-ray diffraction (X-ray) and electron microscopy (Electron microscopy), excluding nuclear magnetic resonance (NMR); 1.2, the resolution is less than 2.5 Å (Ångström); 1.3, the crystal R-factor is greater than 0.25; 1.4, the sequence is between 40 and 10000 amino acids; 1.5, the sequence similarity is less than 50%.

Finally, a total of 26577 single-chained protein structures are obtained using the PDB50 dataset, and 24577, 1000, and 1000 single-chained protein structures are selected as training sets, validation sets, and test sets, respectively.

S2, protein graph representation: The nearest k neighbor amino acids of each amino acid (k is set to 20) are searched to construct the directed edges in the protein structure diagram.

The node features and edge features on the diagram are the three-dimensional space coordinates and dihedral angle information of the backbone atom and the virtual atom, respectively. The dimensions of the node feature and edge feature are 6 and 36, respectively.

The virtual atom CB is constructed based on the bond length, bond angle, and dihedral angle parameters of the protein backbone geometry, the bond length of CC is 1.54 Å, the bond angle of N_CA_CB is 110.6°, and the dihedral angle of C_N_CA_CB is −124.4°.

S3, model architecture: The present disclosure uses a graph neural network algorithm to model the three-dimensional structure information of protein backbone (FIG. 1A), and the overall architecture is shown in FIG. 1B. The graph neural network includes four modules: graph construction, preprocessing layer, graph neural network encoder and decoder (FIG. 1B). The following is a specific implementation process of the four modules:
i) The three-dimensional structure of the backbone structure of the protein is first preprocessed, including removing the side chain atoms and adding virtual atoms to standardize the structural information and construct a protein graph representation suitable for graph neural network processing (FIG. 1A). Subsequently, the protein graph is constructed, with the residue or virtual atom as the node and the spatial or topological relationship between the residues as the edge.
ii) The preprocessing layer includes a 1D convolution layer, four residual blocks, an InstanceNorm layer, and a ReLU activation function, before the graph neural network processing, the node features are first updated by the preprocessing layer, which includes a 1D convolution layer, four residual blocks, an InstanceNorm layer, and a ReLU activation function.
iii) Graph neural network encoder uses a message passing neural network to update the node and edge features in protein structure. The graph neural network encoder consists of five layers of MPNN, each layer of MPNN consists of an edge update module, a graph convolution module, and a residual module. The edge update module includes a 1D convolution layer, two residual blocks, a BatchNorm layer, and a ReLU activation function. The graph convolution module is used to update the node features on the graph, including 1 1D convolution layer, 2 residual blocks, 1 BatchNorm layer, and 1 ReLU activation function. The residual module includes two residual blocks, a BatchNorm layer, and a ReLU activation function, and finally fuses with the updated node features.
iv) The decoder uses multi-layer 1D convolution and residual blocks. Specifically, it includes 1D convolution, 4 residual blocks (hidden layer is 128), InstanceNorm, ReLU, 1D convolution, 4 residual blocks (hidden layer is 64), InstanceNorm, ReLU, 1D convolution (output dimension is 20). S4, model training and performance evaluation: Self-supervised learning is performed using known side-chain amino acid types as labels, and pre-training on the collected single-stranded dataset.

For each protein structure, the graph neural network model outputs a probability of 20 amino acids per position. For each position, the amino acid type with the highest probability is selected as the model prediction result, and the cross-entropy loss is calculated between the predicted amino acid type and the label amino acid type. Using the Adam optimizer, the learning rate is set to 0.002, and the learning rate is adjusted using StepLR. The learning rate is multiplied by gamma (gamma=0.1) each time for a certain training epoch.

In order to further improve the predictive ability of the model for specific protein structures, the present disclosure proposes a method for fine-tuning based on meta-learning strategy, as shown in FIG. 2. The core idea of meta-learning is “learning how to learn”. By training on multiple related tasks, the model can quickly adapt to new but structurally similar protein tasks. In this present disclosure, each task corresponds to a protein single-chain backbone residue type recovery subtask.

Specifically, this method first searches the homologous proteins in the PDB database through Foldseek, and clusters them (similarity is 50%). The single-chain structure similar to the target protein is used as the test set, and the other is used as the training set. The pre-trained graph neural network model is fine-tuned on the database with 50 epochs, and the learning rate is set to 1e⁻⁶ to ensure that the model can effectively generate context-specific representations of the target protein on the test set. Through the meta-learning strategy, the model not only inherits the general protein structure knowledge learned in the pre-training process, but can also quickly adjust the model initial parameters for a new protein structure. The evaluation results show that using the method of the present disclosure, the fine-tuned model shows higher sequence recovery rate and better perplexity on multiple protein test sets than the pre-trained model (FIG. 3). In the evaluation of the three case proteins, the performance of the model is greatly improved after meta-learning fine-tuning (FIG. 4): the sequence recovery rate of TadA8e strand E increases from 0.448 to 0.545, RMSD decreases from 0.774 to 0.683, and the average pLDDT increases from 0.961 to 0.968; the sequence recovery rate of TadA8e strand F increases from 0.455 to 0.545, RMSD increases slightly from 0.677 to 0.699, and the average pLDDT maintains from 0.954 to 0.953. The sequence recovery rate of SpCas9 strand D increases from 0.505 to 5.921, RMSD increases from 0.565 to 3.745, and the average pLDDT increases from 0.885 to 0.886. The sequence recovery rate of OsPHR2 increases from 0.537 to 0.556, the RMSD decreases from 0.674 to 0.365, and the average pLDDT increases from 0.893 to 0.931. These results show that the folding performance and structural prediction reliability of the fine-tuning model on specific proteins are significantly improved. Since the model has a more accurate understanding of the three-dimensional structure of the protein, the preferred mutation sites predicted based on the model have a higher possibility of experimental verification, which is helpful to obtain reliable candidate mutations in protein function enhancement or stability modification.

S5, model inference: The three-dimensional structure of the target gene-edited protein is downloaded from the PDB database. Firstly, the single-chain structure of the target protein is extracted, and the graph neural network model is used for prediction. The output value of the model is converted into a probability distribution using the Softmax function, and then the amino acid type with the highest probability at each position is extracted as the prediction result of the position. According to the predicted probability value, the position of the mutation is sorted, and finally, the potential mutation that can improve the stability of the protein structure is obtained.

### Embodiment 1: Engineering of TadA8e Base Editor

The wheat line used in this embodiment is KN199 (KENONG 199), and wheat lines such as Fielder can also be used.

The specific transformation process is as follows:

### 1, TadA8e Model Fine-Tuning

Firstly, TadA8e (PDB: 6VPC) is used as a query protein structure, and Foldseek is used to search all homologous proteins in the PDB50 database to construct a TadA8e homologous protein database. The database is clustered using Foldseek's easy-cluster clustering algorithm (the clustering threshold is set to 0.5). The cluster of TadA8e is selected as the test set, and other homologous protein structures are selected as the training set. Using the meta-learning strategy, the pre-trained graph neural network model is fine-tuned by 50 epochs on the database, and the learning rate is set to 1e⁻⁶ to obtain the fine-tuned MetaTadA8e model.

### 2, Prediction of TadA8e Beneficial Mutations

Chain E and chain F in TadA8e are extracted and input into the fine-tuned MetaTadA8e model, respectively. The output value of the model is converted into a probability value by the Softmax function, and the final prediction results are sorted according to the probability value. A total of 6 mutation sites with a probability value greater than 90% are obtained in the fine-tuned MetaTadA8e model. Among them, there are 3 mutation sites with strand E structure as input: T83N, H128Y, and Y123H, and 3 mutation sites with strand F structure as input: C141V, V35I, and T83D. As shown in Table 1 and FIG. 5.

TABLE 1
Prediction results of different strands of the TadA8e protein

| | | Predicted site | Wild-type site |
|---|---|---|---|
| Strand | Mutation | probability | probability |
| E | T83N | 0.999584 | 0.000025 |
| E | H128Y | 0.983859 | 0.002105 |
| E | Y123H | 0.917988 | 0.001505 |
| F | C141V | 0.995634 | 0.002166 |
| F | V35I | 0.958744 | 0.041183 |
| F | T83D | 0.956068 | 0.003701 |

### 3, Construction of the TadA8e Original Vector

The TadA8e and SpCas9n sequences optimized for wheat codon preference are obtained by the gene synthesis method, which are SEQ ID NO.1 and SEQ ID NO.2, respectively.

SEQ ID NO. 1:
ATGTCCGAGGTGGAGTTCTCCCACGAGTACTGGATGAGGCACGCCCTC
ACCCTCGCCAAGAGGGCCAGGGACGAGAGGGAGGTGCCAGTGGGCGCC
GTGCTCGTGCTCAACAACAGGGTGATCGGCGAGGGCTGGAACAGGGCC
ATCGGCCTCCACGACCCAACCGCCCACGCCGAGATCATGGCCCTCAGG
CAAGGCGGCCTCGTGATGCAAAACTACAGGCTCATCGACGCCACCCTC
TACGTGACCTTCGAGCCATGCGTGATGTGCGCCGGCGCCATGATCCAC
TCCAGGATCGGCAGGGTGGTGTTCGGCGTGAGGAACTCCAAGAGGGGC
GCCGCCGGCTCCCTCATGAACGTGCTCAACTACCCAGGCATGAACCAC
AGGGTGGAGATCACCGAGGGCATCCTCGCCGACGAGTGCGCCGCCCTC
CTCTGCGACTTCTACAGGATGCCAAGGCAAGTGTTCAACGCCCAAAAG
AAGGCCCAATCCTCCATCAACTGA;
SEQ ID NO. 2:
ATGGACAAGAAGTACTCCATCGGCCTCGCCATCGGCACCAACTCCGTG
GGCTGGGCCGTGATCACCGACGAGTACAAGGTGCCATCCAAGAAGTTC
AAGGTGCTCGGCAACACCGACAGGCACTCCATCAAGAAGAACCTCATC
GGCGCCCTCCTCTTCGACTCCGGCGAGACGGCCGAGGCCACCAGGCTC
AAGAGGACCGCCAGGAGGAGGTACACCAGGAGGAAGAACAGGATCTGC
TACCTCCAAGAGATCTTCTCCAACGAGATGGCCAAGGTGGACGACTCC
TTCTTCCACAGGCTCGAGGAGTCCTTCCTCGTGGAGGAGGACAAGAAG
CACGAGAGGCACCCAATCTTCGGCAACATCGTGGACGAGGTGGCCTAC
CACGAGAAGTACCCAACCATCTACCACCTCAGGAAGAAGCTCGTGGAC
TCCACCGACAAGGCCGACCTCAGGCTCATCTACCTCGCCCTCGCCCAC
ATGATCAAGTTCAGGGGCCACTTCCTCATCGAGGGCGACCTCAACCCA
GACAACTCCGACGTGGACAAGCTCTTCATCCAACTCGTGCAAACCTAC
AACCAACTCTTCGAGGAGAACCCAATCAACGCCTCCGGCGTGGACGCC
AAGGCCATCCTCTCCGCCAGGCTCTCCAAGTCCAGGAGGCTCGAGAAC
CTCATCGCCCAACTCCCAGGCGAGAAGAAGAACGGCCTCTTCGGCAAC
CTCATCGCCCTCTCCCTCGGCCTCACCCCAAACTTCAAGTCCAACTTC
GACCTCGCCGAGGACGCCAAGCTCCAACTCTCCAAGGACACCTACGAC
GACGACCTCGACAACCTCCTCGCCCAAATCGGCGACCAATACGCCGAC
CTCTTCCTCGCCGCCAAGAACCTCTCCGACGCCATCCTCCTCTCCGAC
ATCCTCAGGGTGAACACCGAGATCACCAAGGCCCCACTCTCCGCCTCC
ATGATCAAGAGGTACGACGAGCACCACCAAGACCTCACCCTCCTCAAG
GCCCTCGTGAGGCAACAACTCCCAGAGAAGTACAAGGAGATCTTCTTC
GACCAATCCAAGAACGGCTACGCCGGCTACATCGACGGCGGCGCCTCC
CAAGAGGAGTTCTACAAGTTCATCAAGCCAATCCTCGAGAAGATGGAC
GGCACCGAGGAGCTGCTCGTGAAGCTCAACAGGGAGGACCTCCTCAGG
AAGCAAAGGACCTTCGACAACGGCTCCATCCCACACCAAATCCACCTC
GGCGAGCTGCACGCCATCCTCAGGAGGCAAGAGGACTTCTACCCATTC
CTCAAGGACAACAGGGAGAAGATCGAGAAGATCCTCACCTTCCGCATC
CCATACTACGTGGGCCCACTCGCCAGGGGCAACTCCAGGTTCGCCTGG
ATGACCAGGAAGTCCGAGGAGACGATCACCCCATGGAACTTCGAGGAG
GTGGTGGACAAGGGCGCCTCCGCCCAATCCTTCATCGAGAGGATGACC
AACTTCGACAAGAACCTCCCAAACGAGAAGGTGCTCCCAAAGCACTCC
CTCCTCTACGAGTACTTCACCGTGTACAACGAGCTGACCAAGGTGAAG
TACGTGACCGAGGGCATGAGGAAGCCAGCCTTCCTCTCCGGCGAGCAA
AAGAAGGCCATCGTGGACCTCCTCTTCAAGACCAACAGGAAGGTGACC
GTGAAGCAACTCAAGGAGGACTACTTCAAGAAGATCGAGTGCTTCGAC
TCCGTGGAGATCTCCGGCGTGGAGGACAGGTTCAACGCCTCCCTCGGC
ACCTACCACGACCTCCTCAAGATCATCAAGGACAAGGACTTCCTCGAC
AACGAGGAGAACGAGGACATCCTCGAGGACATCGTGCTCACCCTCACC
CTCTTCGAGGACAGGGAGATGATCGAGGAGAGGCTCAAGACCTACGCC
CACCTCTTCGACGACAAGGTGATGAAGCAACTCAAGAGGAGGAGGTAC
ACCGGCTGGGGCAGGCTCTCCAGGAAGCTCATCAACGGCATCAGGGAC
AAGCAATCCGGCAAGACCATCCTCGACTTCCTCAAGTCCGACGGCTTC
GCCAACAGGAACTTCATGCAACTCATCCACGACGACTCCCTCACCTTC
AAGGAGGACATCCAAAAGGCCCAAGTGTCCGGCCAAGGCGACTCCCTC
CACGAGCACATCGCCAACCTCGCCGGCTCCCCAGCCATCAAGAAGGGC
ATCCTCCAAACCGTGAAGGTGGTGGACGAGCTGGTGAAGGTGATGGGC
AGGCACAAGCCAGAGAACATCGTGATCGAGATGGCCAGGGAGAACCAA
ACCACCCAAAAGGGCCAAAAGAACTCCAGGGAGAGGATGAAGAGGATC
GAGGAGGGCATCAAGGAGCTGGGCTCCCAAATCCTCAAGGAGCACCCA
GTGGAGAACACCCAACTCCAAAACGAGAAGCTCTACCTCTACTACCTC
CAAAACGGCAGGGACATGTACGTGGACCAAGAGCTGGACATCAACAGG
CTCTCCGACTACGACGTGGACCACATCGTGCCACAATCCTTCCTCAAG
GACGACTCCATCGACAACAAGGTGCTCACCAGGTCCGACAAGAACAGG
GGCAAGTCCGACAACGTGCCATCCGAGGAGGTGGTGAAGAAGATGAAG
AACTACTGGAGGCAACTCCTCAACGCCAAGCTCATCACCCAAAGGAAG
TTCGACAACCTCACCAAGGCCGAGAGGGGCGGCCTCTCCGAGCTGGAC
AAGGCCGGCTTCATCAAGAGGCAACTCGTGGAGACGAGGCAAATCACC
AAGCACGTCGCCCAAATCCTCGACTCCAGGATGAACACCAAGTACGAC
GAGAACGACAAGCTCATCAGGGAGGTGAAGGTGATCACCCTCAAGTCC
AAGCTCGTGTCCGACTTCAGGAAGGACTTCCAATTCTACAAGGTGAGG
GAGATCAACAACTACCACCACGCCCACGACGCCTACCTCAACGCCGTG
GTGGGCACCGCCCTCATCAAGAAGTACCCAAAGCTCGAGTCCGAGTTC
GTGTACGGCGACTACAAGGTGTACGACGTGAGGAAGATGATCGCCAAG
TCCGAGCAAGAGATCGGCAAGGCCACCGCCAAGTACTTCTTCTACTCC
AACATCATGAACTTCTTCAAGACCGAGATCACCCTCGCCAACGGCGAG
ATCAGGAAGAGGCCACTCATCGAGACGAACGGCGAGACGGGCGAGATC
GTGTGGGACAAGGGCAGGGACTTCGCCACCGTGAGGAAGGTGCTCTCC
ATGCCACAAGTGAACATCGTGAAGAAGACCGAGGTGCAAACCGGCGGC
TTCTCCAAGGAGTCCATCCTCCCAAAGAGGAACTCCGACAAGCTCATC
GCCAGGAAGAAGGACTGGGACCCAAAGAAGTACGGCGGCTTCGACTCC
CCAACCGTGGCCTACTCCGTGCTCGTGGTGGCCAAGGTGGAGAAGGGC
AAGTCCAAGAAGCTCAAGTCCGTGAAGGAGCTGCTCGGCATCACCATC
ATGGAGAGGTCCTCCTTCGAGAAGAACCCAATCGACTTCCTCGAGGCC
AAGGGCTACAAGGAGGTGAAGAAGGACCTCATCATCAAGCTCCCAAAG
TACTCCCTCTTCGAGCTGGAGAACGGCAGGAAGAGGATGCTCGCCTCC
GCCGGCGAGCTGCAAAAGGGCAACGAGCTGGCCCTCCCATCCAAGTAC
GTGAACTTCCTCTACCTCGCCTCCCACTACGAGAAGCTCAAGGGCTCC
CCAGAGGACAACGAGCAAAAGCAACTCTTCGTGGAGCAACACAAGCAC
TACCTCGACGAGATCATCGAGCAAATCTCCGAGTTCTCCAAGAGGGTG
ATCCTCGCCGACGCCAACCTCGACAAGGTGCTCTCCGCCTACAACAAG
CACAGGGACAAGCCAATCAGGGAGCAAGCCGAGAACATCATCCACCTC
TTCACCCTCACCAACCTCGGCGCCCCAGCCGCCTTCAAGTACTTCGAC
ACCACCATCGACAGGAAGAGGTACACCTCCACCAAGGAGGTGCTCGAC
GCCACCCTCATCCACCAATCCATCACCGGCCTCTACGAGACGAGGATC
GACCTCTCCCAACTCGGCGGCGACTGA.

The pBlunt-UBI-NOS vector is digested with Sac I enzyme, and the homologous arm is designed according to the incision. The synthesized sequence is amplified by primers with homologous arms, and the sequence is connected to pBlunt-UBI-NOS by the seamless cloning method to construct the expression vector pB-UBI-TadA8e-SpCas9n-NOS. The combination of this unit is shown in FIG. 6.

Other elements, such as bpNLS, L (linker), and npNLS are SEQ ID NO.3, SEQ ID NO.4, and SEQ ID NO.5, respectively.

SEQ ID NO. 3:
AAGAGGACCGCCGACGGCTCCGAGTTCGAGTCCCCAAAGAAGAAGAGG
AAGGTG;
SEQ ID NO. 4:
TCCGGCGGCTCCTCCGGCGGCTCCTCCGGCTCCGAGACGCCAGGCACC
TCCGAGTCCGCCACCCCAGAGTCCTCCGGCGGCTCCTCCGGCGGCTCC;
SEQ ID NO. 5:
AAGAGGCCAGCCGCCACCAAGAAGGCCGGCCAAGCCAAGAAGAAGAAG.

### 4, Construction of the TadA8e Mutant Vector

According to the amino acid mutation sites predicted by the above model, the corresponding primers containing point mutations are designed. The point mutations are introduced by PCR, and the fragments are infused into the pBlunt-UBI-NOS vector by seamless cloning to construct the pB-UBI-TadA8e-SpCas9n-NOS vector. A total of 6 vectors of pB-UBI-mTadA8e (V35I)-SpCas9n-NOS, pB-UBI-mTadA8e (T83N)-SpCas9n-NOS, etc., containing single point amino acid mutations are constructed, as shown in FIG. 6.

### 5, Flow Cytometry Analysis of the Editing Efficiency of Mutants

Using the mGFP>GFP screening system (including pB-UBI-mGFP (Q70*)-NOS vector and B-TaU3-tRNA-(mGFP) sgRNA-tRNA; mGFP is prematurely terminated due to Q70* mutation and does not emit fluorescence; when the negative strand produces editing of A>G, the fluorescence is emitted), and 6 single-point amino acid mutations that have been constructed are screened. The experimental method is as follows: Wheat protoplasts are prepared by enzymatic hydrolysis; 10 μg of protein expression vectors (such as B-UBI-TadA8e-SpCas9n-NOS, etc.) and 10 μg of mGFP>GFP screening system (pB-UBI-mGFP (Q70*)-NOS vector and B-TaU3-tRNA-(mGFP) sgRNA-tRNA) are co-transformed into wheat protoplasts by PEG-induced chemical transformation using EndoFree Plasmid Midi Kit (Kangwei Century, Jiangsu, China). The transformed protoplasts are incubated at 23° C. in the dark, after 24 hours, the results are statistically analyzed by flow cytometry, and the results are shown in FIG. 7A.

6, sgRNA Design

The specific sgRNAs designed for wheat endogenous genes TaATX4, TaGW8, and TaDEP1 are designed and ligated into the B-TaU3-tRNA-sgRNA-tRNA vector digested by Bbs/enzyme with T4 ligase to construct B-TaU3-tRNA-(TaGW8) sgRNA-tRNA, B-TaU3-tRNA-(TaATX4) sgRNA-tRNA, and B-TaU3-tRNA-(TaDEP1) sgRNA-tRNA vectors.

### 7, Base Editing Efficiency Verification of the A-to-G Base in the Wheat Endogenous Site

The endogenous target sites of the above mutants are verified in wheat protoplasts (KN199), and the gene editing efficiency is detected. The endogenous target sequence is shown in Table 2.

TABLE 2
Gene editing efficiency verification of the
A-to-G base in wheat endogenous target sequence.

| | | GC |
|---|---|---|
| | Target sequence | content % |
| TaGW8 | CAGAAGAGAGAGAGCACAGTCGG | 50 |
| TaATX4 | ATCATATGCAAGCAGATGCATGG | 40 |
| TaDEP1 | ACGAGCTACATTTACTTGAAGGG | 43 |

Experimental method: Preparation of wheat protoplasts by enzymatic hydrolysis; 10 μg of protein expression vectors (such as B-UBI-TadA8e-SpCas9n-NOS, etc.) and 10 μg of guide RNA expression vectors (such as B-TaU3-tRNA-(TaDEP1) sgRNA-tRNA, etc.) are co-transformed into wheat protoplasts by PEG-induced chemical transformation using EndoFree Plasmid Midi Kit (Kangwei Century, Jiangsu, China). The transformed protoplasts are incubated at 23° C. in darkness. After 48 hours, protoplasts are collected, and genomic DNA is extracted. The editing efficiency of different mutants at TaATX4, TaGW8, or TaDEP1 sites is analyzed and counted by amplicon deep sequencing technology, the results are shown in FIG. 7B.

The second-generation sequencing results show that the average editing rate of wild-type TadA8e is about 6.34%, and the average editing rate of mTadA8e-T83N is 16.91% in the TaGW8 site; the average editing rate of wild-type TadA8e is about 10.67%, and the average editing rate of mTadA8e-T83N is 17.00% in the TaATX4 site; the average editing rate of wild-type TadA8e is about 4.16%, and the average editing rate of TadA8e-T83N is 7.63% in the TaDEP1 site. Compared with the wild type TadA8e, the editing efficiency of mTadA8e-T83N in the three sites is significantly improved, which is about 1.59-2.67 times that of the wild type.

In summary, using the crystal structure of TadA8e as input, a TadA8ePro base editor is created through the MetaTadA8e model. The editor introduces a stable and efficient mutation site (T83N) on the basis of TadA8e, this mutation makes the base editor composed of TadA8e-nCas9 significantly improve the editing efficiency of wheat endogenous genes, and has great application potential in wheat gene editing and breeding.

### Embodiment 2: SpCas9 Protein Engineering

The wheat line used in this embodiment is KN199 (KENONG 199), and wheat lines such as Fielder can also be used.

The specific methods are as follows:

### 1, Fine-Tuning of the SpCas9 Model

Strand D of PDB: 4008 is used as the query protein structure, and Foldseek is used to search all homologous proteins in the PDB50 database to construct SpCas9 homologous protein database. The database is clustered using Foldseek's easy-cluster clustering algorithm (clustering threshold is set to 0.5). The cluster of SpCas9 is selected as the test set, and other homologous protein structures are used as the training set. Using the meta-learning strategy, the pre-trained graph neural network model is fine-tuned by 50 epochs on the database, and the learning rate is set to 1e⁻⁶ to obtain the fine-tuned MetaSpCas9 model.

### 2, Prediction of Beneficial Mutations in SpCas9

Chain D of SpCas9 structure (PDB: 4008) is extracted and input into the fine-tuned MetaSpCas9 model. The output value of the model is converted into a probability value by the Softmax function, and the final prediction results are sorted according to the probability value. A total of 20 mutation sites with a probability value greater than 90% are obtained in the fine-tuned MetaSpCas9 model, which are ranked in descending order of probability: Y5W, I473V, S213C, V1342L, R1114S, L508M, M465V, K434G, S318A, S245A, N88G, L35G, H1311N, S1006L, R425K, D1180G, D499C, R165P, V1083I and Q1221G. As shown in Table 3 and FIG. 8.

TABLE 3
Prediction results of SpCas9 protein

| | Predicted site | Wild-type site |
|---|---|---|
| Mutation | probability | probability |
| Y5W | 0.996176 | 0.001495 |
| I473V | 0.991184 | 0.004980 |
| S213C | 0.986891 | 0.004222 |
| V1342L | 0.981515 | 0.000431 |
| R1114S | 0.975115 | 0.000060 |
| L508M | 0.973940 | 0.015100 |
| M465V | 0.966627 | 0.000280 |
| K434G | 0.957663 | 0.005753 |
| S318A | 0.945093 | 0.051839 |
| S245A | 0.944896 | 0.054905 |
| N88G | 0.938336 | 0.008563 |
| L35G | 0.935833 | 0.001319 |
| H1311N | 0.926084 | 0.011629 |
| S1006L | 0.925474 | 0.000894 |
| R425K | 0.923183 | 0.053608 |
| D1180G | 0.919801 | 0.004594 |
| D499C | 0.917124 | 0.076513 |
| R165P | 0.914185 | 0.04475 |
| V1083I | 0.913929 | 0.075696 |
| Q1221G | 0.912356 | 0.000574 |

### 3, Carrier Construction

The SpCas9 sequence optimized for wheat codon preference is obtained by gene synthesis, denoted as SEQ ID NO.6.

SEQ ID NO. 6:
GACAAGAAGTACTCGATCGGCCTCGATATTGGGACTAACTCTGTTGGC
TGGGCCGTGATCACCGACGAGTACAAGGTGCCCTCAAAGAAGTTCAAG
GTCCTGGGCAACACCGATCGGCATTCCATCAAGAAGAATCTCATTGGC
GCTCTCCTGTTCGACAGCGGCGAGACGGCTGAGGCTACGCGGCTCAAG
CGCACCGCCCGCAGGCGGTACACGCGCAGGAAGAATCGCATCTGCTAC
CTGCAGGAGATTTTCTCCAACGAGATGGCGAAGGTTGACGATTCTTTC
TTCCACAGGCTGGAGGAGTCATTCCTCGTGGAGGAGGATAAGAAGCAC
GAGCGGCATCCAATCTTCGGCAACATTGTCGACGAGGTTGCCTACCAC
GAGAAGTACCCTACGATCTACCATCTGCGGAAGAAGCTCGTGGACTCC
ACAGATAAGGCGGACCTCCGCCTGATCTACCTCGCTCTGGCCCACATG
ATTAAGTTCAGGGGCCATTTCCTGATCGAGGGGGATCTCAACCCGGAC
AATAGCGATGTTGACAAGCTGTTCATCCAGCTCGTGCAGACGTACAAC
CAGCTCTTCGAGGAGAACCCCATTAATGCGTCAGGCGTCGACGCGAAG
GCTATCCTGTCCGCTAGGCTCTCGAAGTCTCGGCGCCTCGAGAACCTG
ATCGCCCAGCTGCCGGGCGAGAAGAAGAACGGCCTGTTCGGGAATCTC
ATTGCGCTCAGCCTGGGGCTCACGCCCAACTTCAAGTCGAATTTCGAT
CTCGCTGAGGACGCCAAGCTGCAGCTCTCCAAGGACACATACGACGAT
GACCTGGATAACCTCCTGGCCCAGATCGGCGATCAGTACGCGGACCTG
TTCCTCGCTGCCAAGAATCTGTCGGACGCCATCCTCCTGTCTGATATT
CTCAGGGTGAACACCGAGATTACGAAGGCTCCGCTCTCAGCCTCCATG
ATCAAGCGCTACGACGAGCACCATCAGGATCTGACCCTCCTGAAGGCG
CTGGTCAGGCAGCAGCTCCCCGAGAAGTACAAGGAGATCTTCTTCGAT
CAGTCGAAGAACGGCTACGCTGGGTACATTGACGGCGGGGCCTCTCAG
GAGGAGTTCTACAAGTTCATCAAGCCGATTCTGGAGAAGATGGACGGC
ACGGAGGAGCTGCTGGTGAAGCTCAATCGCGAGGACCTCCTGAGGAAG
CAGCGGACATTCGATAACGGCAGCATCCCACACCAGATTCATCTCGGG
GAGCTGCACGCTATCCTGAGGAGGCAGGAGGACTTCTACCCTTTCCTC
AAGGATAACCGCGAGAAGATCGAGAAGATTCTGACTTTCAGGATCCCG
TACTACGTCGGCCCACTCGCTAGGGGCAACTCCCGCTTCGCTTGGATG
ACCCGCAAGTCAGAGGAGACGATCACGCCGTGGAACTTCGAGGAGGTG
GTCGACAAGGGCGCTAGCGCTCAGTCGTTCATCGAGAGGATGACGAAT
TTCGACAAGAACCTGCCAAATGAGAAGGTGCTCCCTAAGCACTCGCTC
CTGTACGAGTACTTCACAGTCTACAACGAGCTGACTAAGGTGAAGTAT
GTGACCGAGGGCATGAGGAAGCCGGCTTTCCTGTCTGGGGAGCAGAAG
AAGGCCATCGTGGACCTCCTGTTCAAGACCAACCGGAAGGTCACGGTT
AAGCAGCTCAAGGAGGACTACTTCAAGAAGATTGAGTGCTTCGATTCG
GTCGAGATCTCTGGCGTTGAGGACCGCTTCAACGCCTCCCTGGGGACC
TACCACGATCTCCTGAAGATCATTAAGGATAAGGACTTCCTGGACAAC
GAGGAGAATGAGGATATCCTCGAGGACATTGTGCTGACACTCACTCTG
TTCGAGGACCGGGAGATGATCGAGGAGCGCCTGAAGACTTACGCCCAT
CTCTTCGATGACAAGGTCATGAAGCAGCTCAAGAGGAGGAGGTACACC
GGCTGGGGGAGGCTGAGCAGGAAGCTCATCAACGGCATTCGGGACAAG
CAGTCCGGGAAGACGATCCTCGACTTCCTGAAGAGCGATGGCTTCGCG
AACCGCAATTTCATGCAGCTGATTCACGATGACAGCCTCACATTCAAG
GAGGATATCCAGAAGGCTCAGGTGAGCGGCCAGGGGGACTCGCTGCAC
GAGCATATCGCGAACCTCGCTGGCTCGCCAGCTATCAAGAAGGGGATT
CTGCAGACCGTGAAGGTTGTGGACGAGCTGGTGAAGGTCATGGGCAGG
CACAAGCCTGAGAACATCGTCATTGAGATGGCCCGGGAGAATCAGACC
ACGCAGAAGGGCCAGAAGAACTCACGCGAGAGGATGAAGAGGATCGAG
GAGGGCATTAAGGAGCTGGGGTCCCAGATCCTCAAGGAGCACCCGGTG
GAGAACACGCAGCTGCAGAATGAGAAGCTCTACCTGTACTACCTCCAG
AATGGCCGCGATATGTATGTGGACCAGGAGCTGGATATTAACAGGCTC
AGCGATTACGACGTCGATCATATCGTTCCACAGTCATTCCTGAAGGAT
GACTCCATTGACAACAAGGTCCTCACCAGGTCGGACAAGAACCGGGGC
AAGTCTGATAATGTTCCTTCAGAGGAGGTCGTTAAGAAGATGAAGAAC
TACTGGCGCCAGCTCCTGAATGCCAAGCTGATCACGCAGCGGAAGTTC
GATAACCTCACAAAGGCTGAGAGGGGGGGGCTCTCTGAGCTGGACAAG
GCGGGCTTCATCAAGAGGCAGCTGGTCGAGACACGGCAGATCACTAAG
CACGTTGCGCAGATTCTCGACTCACGGATGAACACTAAGTACGATGAG
AATGACAAGCTGATCCGCGAGGTGAAGGTCATCACCCTGAAGTCAAAG
CTCGTCTCCGACTTCAGGAAGGATTTCCAGTTCTACAAGGTTCGGGAG
ATCAACAATTACCACCATGCCCATGACGCGTACCTGAACGCGGTGGTC
GGCACAGCTCTGATCAAGAAGTACCCAAAGCTCGAGAGCGAGTTCGTG
TACGGGGACTACAAGGTTTACGATGTGAGGAAGATGATCGCCAAGTCG
GAGCAGGAGATTGGCAAGGCTACCGCCAAGTACTTCTTCTACTCTAAC
ATTATGAATTTCTTCAAGACAGAGATCACTCTGGCCAATGGCGAGATC
CGGAAGCGCCCCCTCATCGAGACGAACGGCGAGACGGGGGAGATCGTG
TGGGACAAGGGCAGGGATTTCGCGACCGTCAGGAAGGTTCTCTCCATG
CCACAAGTGAATATCGTCAAGAAGACAGAGGTCCAGACTGGCGGGTTC
TCTAAGGAGTCAATTCTGCCTAAGCGGAACAGCGACAAGCTCATCGCC
CGCAAGAAGGACTGGGATCCGAAGAAGTACGGCGGGTTCGACAGCCCC
ACTGTGGCCTACTCGGTCCTGGTTGTGGCGAAGGTTGAGAAGGGCAAG
TCCAAGAAGCTCAAGAGCGTGAAGGAGCTGCTGGGGATCACGATTATG
GAGCGCTCCAGCTTCGAGAAGAACCCGATCGATTTCCTGGAGGCGAAG
GGCTACAAGGAGGTGAAGAAGGACCTGATCATTAAGCTCCCCAAGTAC
TCACTCTTCGAGCTGGAGAACGGCAGGAAGCGGATGCTGGCTTCCGCT
GGCGAGCTGCAGAAGGGGAACGAGCTGGCTCTGCCGTCCAAGTATGTG
AACTTCCTCTACCTGGCCTCCCACTACGAGAAGCTCAAGGGCAGCCCC
GAGGACAACGAGCAGAAGCAGCTGTTCGTCGAGCAGCACAAGCATTAC
CTCGACGAGATCATTGAGCAGATTTCCGAGTTCTCCAAGCGCGTGATC
CTGGCCGACGCGAATCTGGATAAGGTCCTCTCCGCGTACAACAAGCAC
CGCGACAAGCCAATCAGGGAGCAGGCTGAGAATATCATTCATCTCTTC
ACCCTGACGAACCTCGGCGCCCCTGCTGCTTTCAAGTACTTCGACACA
ACTATCGATCGCAAGAGGTACACAAGCACTAAGGAGGTCCTGGACGCG
ACCCTCATCCACCAGTCGATTACCGGCCTCTACGAGACGCGCATCGAC
CTGTCTCAGCTCGGGGGCGAC.

The pBlunt-UBI-NOS vector is digested with Sac I and Kpn I endonucleases, and the homologous arm is designed according to the incision. The synthesized sequence is amplified by primers with homologous arms, and the sequence is connected to the pBlunt-UBI-NOS vector by the seamless cloning method to construct the expression vector pB-UBI-SpCas9-NOS. The combination of this unit is shown in FIG. 9.

The sequences of other elements 3×Flag, NLS, and bpNLS are SEQ ID NO.7, SEQ ID NO.8, and SEQ ID NO.9, respectively.

SEQ ID NO. 7:
GATTACAAGGACCACGACGGGGATTACAAGGACCACGACATTGAT
TACAAGGATGATGATGACAAG;
SEQ ID NO. 8:
ATGGCTCCGAAGAAGAAGAGGAAGGTTGGCATCCACGGGTGCCAG
CTGCT;
SEQ ID NO. 9:
AAGCGGCCAGCGGCGACGAAGAAGGCGGGGCAGGCGAAGAAGAAG
AAG.

According to the predicted amino acid mutation sites, the corresponding primers containing point mutations are designed. The point mutations are introduced by PCR, and the fragments are ligated to the pBlunt-UBI-NOS vector using seamless cloning to construct 20 vectors containing single-point amino acid mutations.

4, sgRNA Design

The specific sgRNAs for wheat endogenous genes TaLOX2, TaPIN1, and TaGW2 are designed and ligated to the B-TaU3-tRNA-sgRNA-tRNA vector digested by Bbs I with T4 ligase.

### 5, Editing Efficiency Verification

The endogenous target sites of the above mutants are verified in wheat protoplasts (KN199) to explore their gene editing efficiency, the endogenous target sequence is shown in Table 4.

TABLE 4
Selected wheat endogenous target sequences
for SpCas9 gene editing efficiency validation

| | | GC |
|---|---|---|
| Target sequence | | content % |
| TaLOX2 | GTGCCGCGCGACGAGCTCTT | 70 |
| TaPIN1 | TCACCGTGGGCGCCGCCACC | 80 |
| TaGW2 | CCAGGATGGGGTATTTCTAG | 50 |

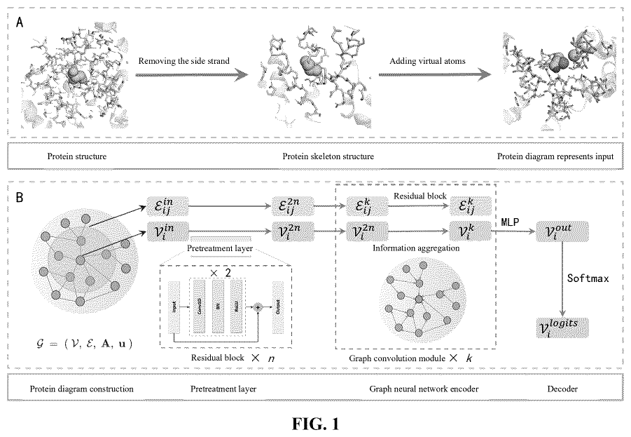

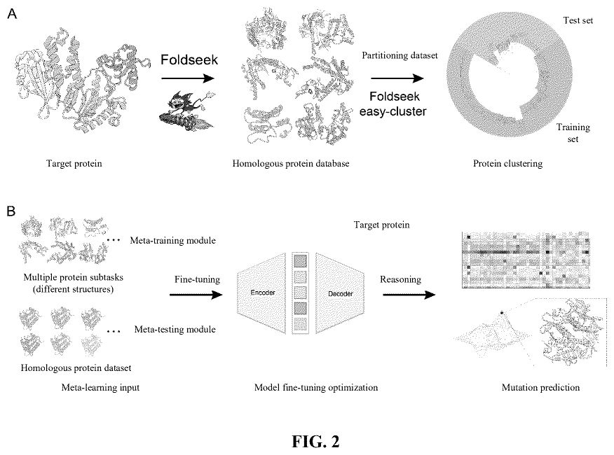

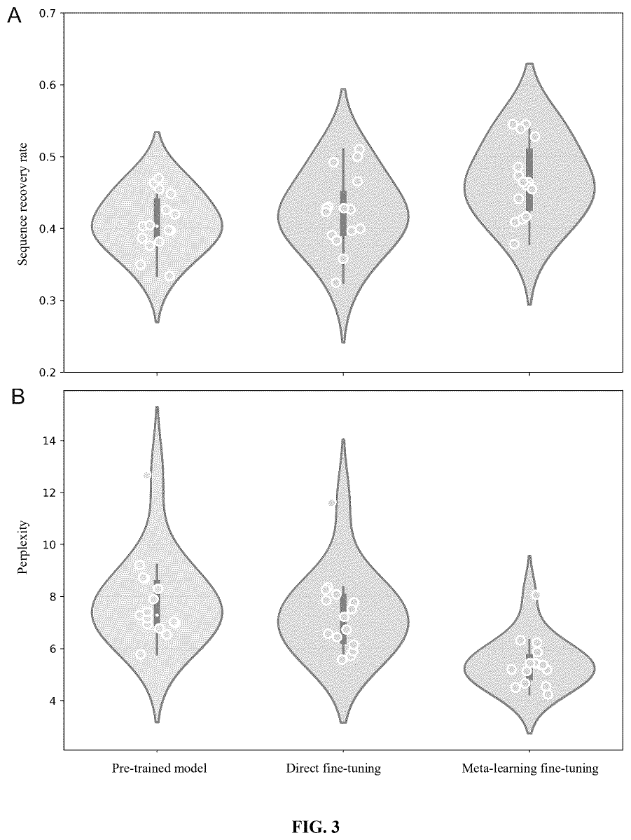

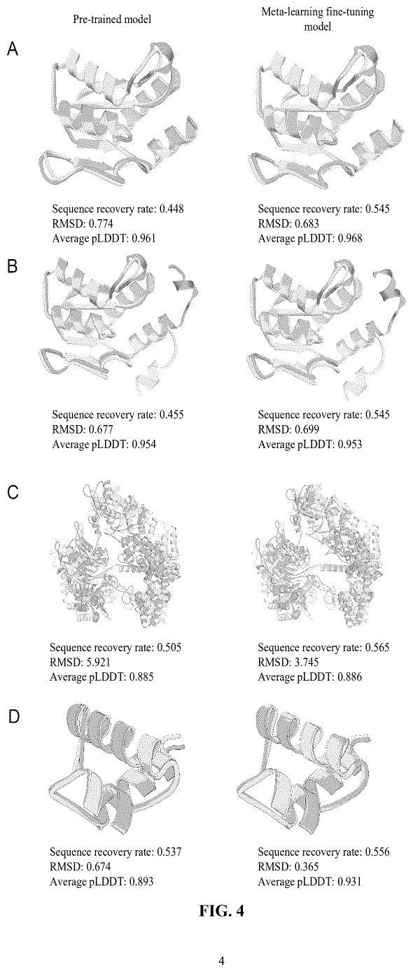

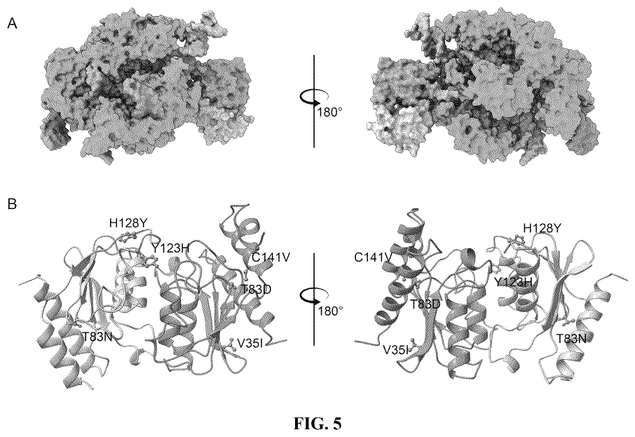

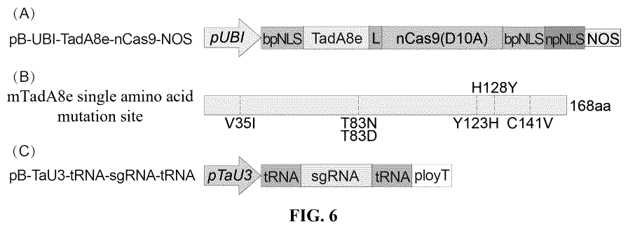

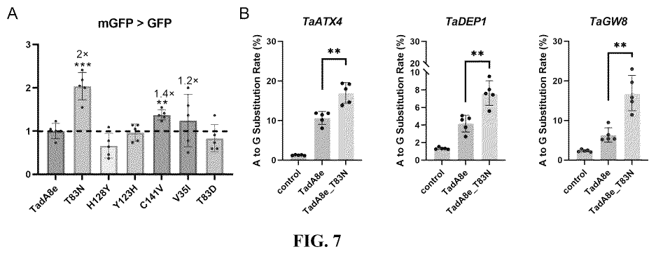

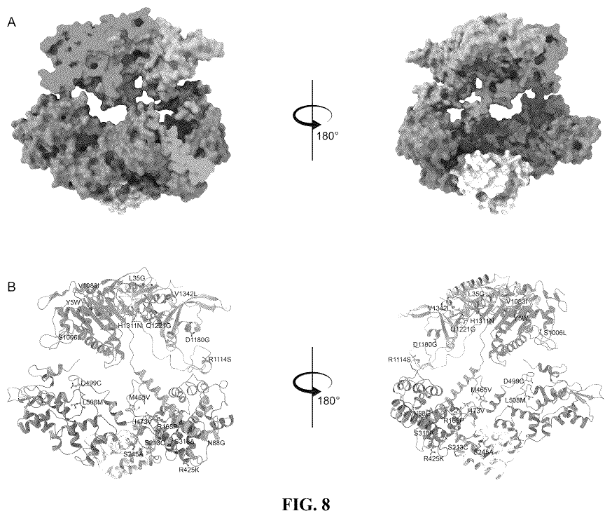

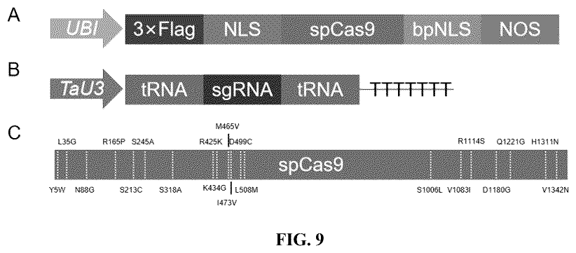

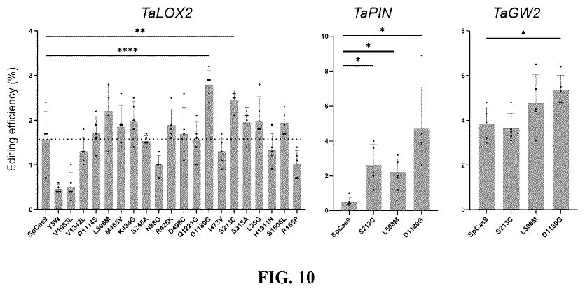

Experimental methods: Preparation of wheat protoplasts by enzymatic hydrolysis; using EndoFree Plasmid Midi Kit (Kangwei Century, Jiangsu, China), 10 μg of protein expression vector and 10 μg of guide RNA expression vector are co-transformed into wheat protoplasts by PEG-induced chemical transformation method. The transformed protoplasts are incubated at 23° C. in the dark. After 48 hours, the protoplasts are collected, and the genomic DNA is extracted. Using the amplicon deep sequencing technology, the editing efficiency of different mutants at the target site is analyzed and counted. The results are shown in FIG. 10.

The second-generation sequencing results show that the average editing rate of the original SpCas9 protein is 1.58%, and the average editing rate of mSpCas9-D1180G is 2.8% in the TaLOX2 site; the average editing rate of the original SpCas9 protein is 0.52%, and the average editing rate of mSpCas9-D1180G is 4.72% in the TaPIN1 site; the average editing rate of the original SpCas9 protein is 3.84%, and the average editing rate of mSpCas9-D1180G is 5.36% in the TaGW2 site. Compared with the wild type SpCas9, the editing efficiency of mSpCas9-D1180G in the three sites is significantly improved, which is 1.39-9.07 times that of the wild type, as shown in FIG. 10.

In summary, using the crystal structure of SpCas9 as input, the mSpCas9-D1180G variant (Cas9Plus) is created through the MetaSpCas9 model. Cas9Plus introduces a stable and efficient mutation site (D1180G) on the basis of SpCas9, this mutation significantly improves the editing efficiency of SpCas9 editing protein on wheat endogenous genes, and has greater application potential in wheat gene editing breeding.

### Embodiment 3: Preliminary Screening of High-Activity OsPHR2 Transcription Factor

### 1, Structure Prediction and Selection of the OsPHR2 Transcription Factor

AlphaFold is used to predict the protein structure of OsPHR2. The average pLDDT of the predicted structure is 44.63, and the average pLDDT of Ca is 47.14. The core structure region (249-302) is selected, and the average pLDDT of the selected structure is 89.97, and the average pLDDT of Ca is 95.13. The above selected structure is used as the input of the following model to achieve the prediction of beneficial mutations.

### 2, Fine-Tuning of the OsPHR2 Model

The selected OsPHR2 is used as the query protein structure, and Foldseek is used to search all homologous proteins in the PDB50 database to construct the OsPHR2 homologous protein database. The database is clustered using Foldseek's easy-cluster clustering algorithm (clustering threshold is set to 0.5). The cluster of OsPHR2 is selected as the test set, and other homologous protein structures are used as the training set. Using the meta-learning strategy, the pre-trained graph neural network model is fine-tuned on the database by 50 epochs, and the learning rate is set to 1e⁻⁶ to obtain a fine-tuned MetaOsPHR2 model.

### 3, Prediction of Beneficial Mutations in OsPHR2

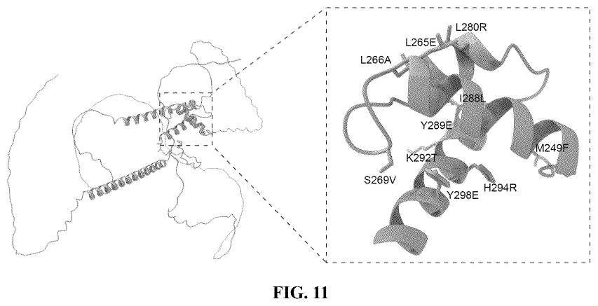

The core structure of the selected OsPHR2 transcription factor is extracted and input into the fine-tuned MetaOsPHR2 model, the output value of the model is converted into a probability value by the Softmax function, and the final prediction results are sorted according to the probability value. A total of 10 mutation sites with the highest scores are screened in the fine-tuned MetaOsPHR2 model: S269V, L266A, H294R, I288L, L280R, K292T, M249F, Y298L, Y289E, L265E, as shown in Table 5 and FIG. 11. These mutation sites are located in the high confidence region of the predicted transcription factor structure, providing candidate mutation information for subsequent experimental verification.

TABLE 5
Prediction results of the OsPHR2 transcription factor

| | Predicted site | Wild-type site |
|---|---|---|
| Mutation | probability | probability |
| S269V | 0.914654 | 0.004482 |
| L266A | 0.855959 | 0.002309 |
| H294R | 0.823817 | 0.009759 |
| I288L | 0.777647 | 0.151182 |
| L280R | 0.748582 | 0.030551 |
| K292T | 0.520733 | 0.082723 |
| M249F | 0.518263 | 0.129901 |
| Y298L | 0.486338 | 0.08538 |
| Y289E | 0.459831 | 0.010832 |
| L265E | 0.405171 | 0.021155 |

### 4, Construction of the OsPHR2 Mutant Vector

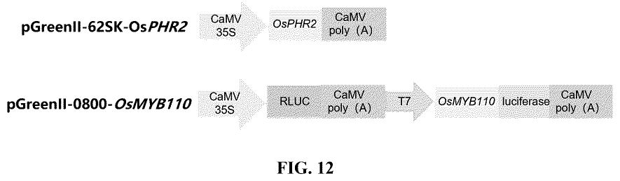

The corresponding point mutation primers are designed according to the prediction results, and the point mutation is introduced into the OsPHR2 gene sequence by PCR. Subsequently, the mutant fragment is ligated to the pGreenII-62SK vector by seamless cloning technology, and the promoter sequence of the downstream gene OsMYB110 is ligated to the pGreenII-0800 vector to construct pGreenII-62SK-OsPHR2 and pGreenII-0800-OsMYB110 vectors, respectively. Finally, 10 single-point mutation vectors are obtained, such as pGreenII-62SK-OsPHR2-H294R, pGreenII-62SK-OsPHR2-L266A, etc. (as shown in FIG. 12).

The promoter sequences of OsPHR2 and OsMYB110 are SEQ ID NO. 10 and SEQ ID NO.11, respectively.

SEQ ID NO. 10:
ATGGAGAGAATAAGCACCAATCAGCTCTACAATTCTGGAATTCCGGTG
ACTGTGCCATCGCCTCTGCCTGCTATACCAGCTACCCTGGATGAAAAC
ATTCCCAGGATTCCAGATGGGCAGAATGTTCCGCGGGAGAGAGAATTG
AGAAGCACACCTATGCCACCTCATCAGAATCAGAGTACTGTTGCTCCT
CTTCATGGGCATTTTCAGTCCAGTACCGGGTCTGTTGGGCCTCTGCGT
TCGTCCCAGGCGATAAGGTTCTCTTCAGTTTCAAGCAATGAGCAATAT
ACAAATGCCAATCCTTACAATTCTCAACCGCCGAGTAGTGGGAGTTCT
TCAACGCTCAATTATGGATCACAATATGGAGGCTTTGAACCTTCCTTG
ACTGATTTTCCAAGAGATGCTGGGCCGACGTGGTGTCCTGATCCAGTT
GATGGCTTGCTTGGATATACAGATGATGTCCCTGCTGGGAACAATTTG
ACTGAAAACAGTTCTATTGCAGCTGGTGATGAACTTGCCAAGCAAAGT
GAATGGTGGAATGATTTTATGAATTATGACTGGAAAGATATTGATAAC
ACAGCTTGTACTGAAACTCAACCACAGGTTGGACCAGCTGCGCAATCA
TCTGTCGCAGTTCACCAATCAGCTGCCCAACAATCAGTTTCATCTCAA
TCAGGAGAACCTTCTGCAGTTGCTATACCCTCGCCCTCTGGTGCCTCC
AATACCTCCAACTCCAAGACACGAATGAGATGGACTCCTGAACTTCAT
GAGCGCTTTGTAGATGCTGTCAATCTACTTGGTGGCAGTGAAAAAGCT
ACTCCCAAGGGTGTGTTAAAGCTAATGAAGGCAGACAATTTGACCATT
TATCATGTTAAAAGTCACCTTCAGAAATACAGAACAGCTCGATACAGA
CCAGAATTGTCTGAAGGTTCTTCAGAAAAGAAGGCAGCCTCAAAAGAG
GACATACCATCAATAGATCTGAAAGGAGGGAACTTTGATCTCACTGAG
GCATTGCGTCTCCAGTTAGAACTCCAAAAGAGGCTTCATGAACAGCTT
GAGATCCAAAGAAGTTTGCAGCTGAGAATTGAGGAGCAAGGGAAGTGC
CTTCAGATGATGCTCGAGCAGCAGTGCATACCTGGGACAGACAAGGCG
GTGGATGCTTCAACCTCAGCAGAAGGAACAAAGCCATCTTCTGATCTT
CCAGAATCTTCTGCCGTGAAGGATGTTCCAGAGAACAGTCAGAACGGA
ATAGCCAAACAAACAGAATCAGGTGACAGATAA
SEQ ID NO. 11:
CCAATTAGCCCAGCCTGGTGTTAATTAGCTGGATGACTGGATCTTACT
ATACATGGCAAAAGTGTTCACCACTTTGATGTCAATTATTGGAGAGTT
AATTACCCATATATATGCGTAGTATATGTGATTTTGAAAGTGTCCAAA
CATGTAGTGCAATTTTATTGGGAGTAATTAATACACTGAATTAAAATT
CATAAAAGAAAGATAAGGTGTTACCAGGTCAGAGATTTTACTTTACTT
AAATACCACATAGCAATGTGAATACGTGTGGTGAAACTATACCACTTT
GATTTATGGACAAAGTTACTGATGATAGTTACACTAAAACTAAATAAT
GCAATCAACATGGCCTCAGTAACATGGATAAAAAACTACTAAATTATT
ATTGCCGAAAGTAATTGGGTGACTTCGTCAAGATCTTACTGTTGTACG
TGAAGTGTGAACAGTACCGTACCGTCTAATTTTATAAAGGATGCAGCG
TGAGACGGGTATATTAACCACTAACTCGCACTAGGACGGCTTATCAAC
CATTTACAATAAAGCATTAAAGCCTTCTTCATAGTGGAGAAATGTGAA
AGCACTTTTAAAGAAATTACGCCAAACTATATAAAATTCTTACGTTGT
AAGAAGCCCCAAATATGTATGATTCACTGATTCACACAGCATTGGATG
ATGATTTAGATCTCTCTGATTTAAGTTAGGTGACTTTAAAGACACTAA
CATGTGGAAGATATGGATCCTTCCTTTTCCTCGTAATAAACCATCACA
TAAATAAAACTAACCATCCTAAAGCCTCAACAATCGTGAAAAACTGTA
GATATAGTTCTTGGAAAATTCATATCTTTCTTTCGGAATTACAAAACT
AGAAAAAAAATACTCCCATCGTTTTAAAATATAAGTATTTCTGGTTAT
GAATCTGGACAAGTGTTTATCTAGATTCATAGTTAAAAGTTGTTATAT
TTTAAGATAATGTAGTGCTTATTAGAAAGACATTACATCTTTTCCACA
AAGACTTTTCTTTTTTTACTATGAATTTGAATAAGTATTTCTCTAGGT
GGATATCCTAAAATGAAATACTCTATTCGTCTCAAATATAGCAACTTA
ATACAACATTAGACACCACTTATTAATATGAATCTGGATAGGGATAAC
GAATCTAGACATGATTCATGGCACTAGGTTATATCTATTTTATTTTAG
TTACCGTTATAGTACCTTCTCTATCTTAAAAAACAAATCATGTTCAGA
TTTATAGCACTGGGATGCATCACATCCCGTAGTAGTTTATTTTTATGG
GACGAAAAGAGCACATCAGAATCATGTGCTTTGAAAAAGATCAAAAAC
AAAAAAAAAGAACATCCAAAGGCAAATTCCTTCTTGGGTACAACCATG
TACTCTAGTCCTACAAAGTACCACATAATTCTTGCCACTTGCCATCTC
TTCCCTCTCCCTCCCCATTTGTTCGATTCCCCATTTGGCCTTTTCCTA
GAACCATCCTCCCTCCCCCACAAAACCCCCCAAAAAAATTACAACAAA
AGCAAAATGGATTTGAACAAAATTCAGGATGAAACCTTGAATTCAACA
CTGCACCCTCCTACTAGTAGTAGCACCTCTACCAGTTACTTCTCAATC
CGTACCAAAATATAAACACTTCTAAAATAATATCAAGCCAAATATTTT
TTAACTTTGATTATTAATAGAAAAAAAATAAAAACAAATCAATCATGT
AAAATTGATATTTACTAGATTTATCATTAAACAACTATCATGCTCCAT
ATGTAACTTTTTTTATTTTAAACATCGTACTTTTATAGATATTATTAG
TCAAAGTAGTATCTCGAAGACTAAGTGTAAAATTGTTTATATTTTAGA
GCGGGGAGAGAGAGCTACCCATCTTCATCAGCTAATGATCCAAAAGAG
GCACCAAAAAGAAGAAGGAAGAAAAAAACACGAAACGCGCAGTCGCGT
CTCACCCCCATTTGCCGCACGTTGCCCAACTCCTCCTCCTCCTCGTCA
TCGTCTCCGTTCCGATCCGCGCCCATAAATACGCGCCACCCCGCCCCC
AACCTCGCCGTCCTTGTCCCCCCCAAGAACCCCCCGTGCGCCACCACC
ACCACCACCACCACCACCACCACCACCGAGGAATTCTCGCTGTCGCCG
CCGCCGACGACGACGAGGAGAAGGAGTATCGCTCACAATCTTCCGGGC
CGATGGGGAGGGCGCCGTGCTGCGAGAAGGAGGGGCTGAGGAGAGGGG
CGTGGAGCCCCGAGGAGGACGACCGCCTCGTCGCCTACATCCGCCGCC
ACGGCCACCCCAACTGGCGCGCGCTCCCCAAGCAAGCCGGTTAGTAGT
AGCCTCCGCCGCCGCCGCCGCCGCCGTTGCTGTTGTTCTTGGGTTGAT
GATGATGATGAGATGAGATCGGTGTTGGTTGGTTGCAGGGCTTCTCCG
CTGCGGGAAGAGCTGCAGGCTGCGGTGGATCAACTACCTCCGGCCGGA
CATCAAGCGGGGGAACTTCACCGCCGACGAGGAGGACCTCATCGTCCG
CCTCCACAACTCCCTCG.

### 5, Double Luciferase Reporter Assay of OsPHR2

In rice protoplasts (Nipponbare), 10 single amino acid mutants predicted by the model are screened for dual luciferase reporter genes. The experimental methods are as follows:

Protoplasts are prepared by enzymatic hydrolysis, and 10 μg of protein expression vector (pGreenII-62SK-OsPHR2 and its mutants pGreenII-62SK-OsPHR2-H294R, pGreenII-62SK-OsPHR2-L266A, etc.) and 10 μg of reporter vector pGreenII-0800-OsMYB110 are co-transformed into rice protoplasts by PEG-induced chemical transformation. The transformed protoplasts are incubated at 28° C. in darkness. After 12 h, the protoplasts are collected, and the cells are lysed, the binding of different mutation sites to the downstream promoter is analyzed by the dual luciferase reporter system.

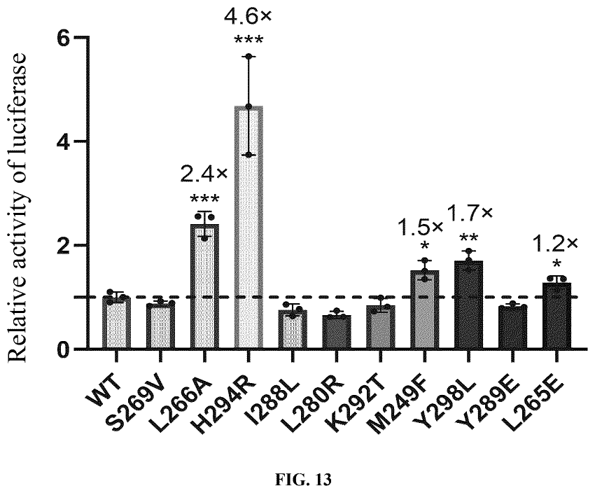

The results are shown in FIG. 13, and the luciferase activity of the 10 single amino acid mutation sites predicted by the model is quantitatively analyzed. The results show that five mutations significantly improved the activation efficiency. Among them, the luciferase activity of the H294R mutant is about 4.6 times higher than that of the wild type, and other highly active mutation sites, such as L265E, L266A, and Y298L, also show different degrees of enhancement (about 1.2-2.4 times). The statistical significance of different numbers of asterisk markers in the map is: “*” (p<0.05); “**” (p<0.01); “***” (p<0.001), indicating that these mutation sites have reliability and repeatability for the enhancement of downstream promoter binding activity.

Although the embodiment gives a detailed description of the present disclosure, for technicians in this field, the technical scheme of the embodiment can be modified, or some of the technical features can be equivalently replaced. Any modification, equivalent replacement, and improvement made within the spirit and principle of the present disclosure are included in the scope of protection of the present disclosure.

This application contains a Sequence Listing XML as a separate part of the disclosure, which presents nucleotide and/or amino acid sequences and associated information using the symbols and format in accordance with the requirements of 37 CFR-1.831-1.835. The XML file named “CNUS-SZ-U-122-2026_SEQ.xml”, created Feb. 6, 2026, 23,337 bytes in size, is submitted herewith and is incorporated by reference in its entirety.

What is claimed is:

1. A protein engineering and directed evolution method based on graph deep learning, comprising the following steps:
S1, construction of a protein structural dataset: using a PISCES server to construct a PDB50 dataset by applying screening conditions item by item;
S2, protein graph representation and feature encoding: searching for nearest k neighbor amino acids of each amino acid, wherein k is set to 20, thereby constructing a directed edge in a protein graph;
S3, establishment of graph neural network model architecture: using a graph neural network algorithm to model three-dimensional structure information of a protein backbone structure;
S4, model training and performance evaluation: performing self-supervised learning using known side chain amino acid types as labels, pre-training on a collected single-chained protein structure dataset; and
S5, model inference: downloading a three-dimensional structure of a target protein from a PDB database; extracting a single-chained structure of the target protein, using a graph neural network model for prediction and using a Softmax function to convert output logits into a probability distribution; extracting an amino acid type with a higher probability of each position as a prediction result of the position, sorting mutation positions according to a predicted probability, and finally obtaining a potential mutation that can improve a property of a protein.

2. The protein engineering and directed evolution method based on graph deep learning according to claim 1, wherein in S1, the screening conditions are as follows:
structure determination methods comprise X-ray diffraction and electron microscopy, and exclude nuclear magnetic resonance;
a resolution is less than 2.5 Å;
a crystal R-factor is greater than 0.25;
a sequence is between 40 and 10000 amino acids; and
a sequence similarity is less than 50%.

3. The protein engineering and directed evolution method based on graph deep learning according to claim 1, wherein in S2, node features and edge features on the graph are three-dimensional spatial coordinates of backbone atoms, virtual atoms, and dihedral angle information, and wherein the dimensions of node features and edge features are 6 and 36, respectively.

4. The protein engineering and directed evolution method based on graph deep learning according to claim 3, wherein a virtual atom Cp is constructed according to bond length, bond angle and dihedral angle parameters of a protein backbone geometry, a bond length of CC is 1.54 Å, a bond angle of N_CA_CB is 110.6°, and a dihedral angle of C_N_CA_CB is −124.4°.

5. The protein engineering and directed evolution method based on graph deep learning according to claim 3, wherein in S3, the graph neural network algorithm comprises a graph neural network encoder and a graph neural network decoder.

6. The protein engineering and directed evolution method based on graph deep learning according to claim 5, wherein the feature is that the graph neural network encoder comprises five layers of MPNN, and each layer of MPNN consists of an edge update module, a graph convolution module, and a residual module;
wherein the edge update module comprises a 1D convolution layer, two residual blocks, a BatchNorm layer, and a ReLU activation function;
wherein the graph convolution module is used to update the node features on the graph, comprising 1 1D convolution layer, 2 residual blocks, 1 BatchNorm layer, and 1 ReLU activation function;
the residual module comprises two residual blocks, a BatchNorm layer, and a ReLU activation function, and finally fuses with updated node features.

7. The protein engineering and directed evolution method based on graph deep learning according to claim 5, wherein the graph neural network decoder adopts a multi-layer 1D convolution and residual block, specifically comprising 1D convolution, 4 residual blocks, InstanceNorm, ReLU, 1D convolution, 4 residual blocks, InstanceNorm, ReLU, and 1D convolution.

8. The protein engineering and directed evolution method based on graph deep learning according to claim 1, wherein in S4, for each protein structure, the graph neural network model outputs a probability of 20 amino acids at each position; for each position, the amino acid type with a highest probability is selected as a prediction result of the model, and a cross entropy loss is calculated between a predicted amino acid type and the label amino acid type; using an Adam optimizer, the learning rate is set to 0.002, and the learning rate is adjusted using StepLR, the learning rate is multiplied by gamma each time for a certain training rounds, gamma-0.1.

9. The protein engineering and directed evolution method based on graph deep learning according to claim 8, wherein searching homologous proteins in the PDB database through Foldseek and clustering the homologous proteins, a similarity is 50%; the single-stranded structure of the target protein is used as a test set, and the other is used as a training set, a pre-trained graph neural network model is fine-tuned on the database by 50 Epochs, a learning rate is set to 1e⁻⁶, and finally a performance of the model is evaluated on the test set.

10. An application of the protein engineering and directed evolution method based on graph deep learning according to claim 1, wherein it is used for the engineering of TadA8e base editor, SpCas9 protein engineering, and OsPHR2 transcription factor engineering.
